(19) United States
(12) Patent Application Publication
(10) Pub. No.: US 2002/0166786 A1
(43) Pub. Date: Nov. 14, 2002

(54) Apparatus for storing and transporting cellular matrices

(76) Inventors: Samuel S. Asculai, Toronto (CA); Bruno M. Giannetti, Bonn (DE)

Correspondence Address: MORGAN, LEWIS & BOCKIUS LLP, 1701 MARKET STREET, PHILADELPHIA, PA, 19103-2921, US

(21) Appl. No.: 09/852,241

(22) Filed: May 9, 2001

(51) Int. Cl. A61L 15/00; A61B 17/06

(52) U.S. Cl. 206/438

(57) ABSTRACT

An apparatus comprising a first frame and a second frame adjacent to the first frame, for storing, transporting, and maintaining a biomaterial. In one embodiment, an inner wall of the first frame lockingly engages an outer wall of the second frame. In another embodiment, the apparatus includes a container for holding the first frame and second frame.

### FIELD OF INVENTION

The present invention relates to an apparatus to store, transport, and maintain biomaterials used in medical procedures such as transplants.

### BACKGROUND OF THE INVENTION

More than 500,000 arthroplastic procedures and total joint replacements are performed each year in the United States. Approximately the same number of similar procedures is performed in Europe. Included in this number are about 90,000 total knee replacements and around 50,000 procedures to repair defects in the knee. These numbers are essentially the same in the U.S. (In: Praemer A., Furner S., Rice, D. P., Musculoskeletal Conditions in the United States, American Academy of Orthopaedic Surgeons, Park Ridge, Ill., 1992, 125).

Several techniques have been developed to treat the cartilage structure in joints and to repair the cartilage defects. The articular chondrocytes are specialized mesenchymal derived cells found exclusively in cartilage. Cartilage is an avascular tissue which has physical properties that depend on the extracellular matrix produced by the chondrocytes. Despite the advances in cultivating chondrocytes and manipulating bone and cartilage and the recent development of new biomaterials such as cell-free membranes, no satisfactory and effective method has been presented so far to store, transport and maintain biomaterials such as Chondro-Gide™ sold by Ed Geistlich Sohne (Switzerland).

### SUMMARY OF THE INVENTION

The present invention is an apparatus used to store, transport, and maintain biomaterials. The apparatus includes a first frame adjacent a second frame, and having a center storage area for holding a biomaterial, and optionally a container in which a first frame and second frame are placed. In one embodiment of the invention, the apparatus includes a first frame and the second frame in a side by side arrangement. In another embodiment, the second frame is within the second frame. The first and second frames can have any shape suitable for holding a biomaterial.

### BRIEF DESCRIPTION OF THE DRAWINGS

The invention is best understood from the following detailed description when read in connection with the accompanying figures.

FIG. 1 is a top plan view of one embodiment of the present invention, showing a first frame and a second frame, where the second frame is within the first frame.

FIG. 2 is a side sectional view of the embodiment shown in FIG. 1.

FIG. 3 is a partial perspective view of the embodiment of FIG. 1, where the inner and outer walls of the first and second walls are parallel and angled in the same direction.

FIG. 4 is a partial perspective view of the embodiment of FIG. 1, where the inner and outer walls of the first and second frames are parallel and not angled.

FIG. 5 is a partial perspective view of the embodiment of FIG. 1, where the inner and outer walls of the first and second frames are not parallel and are angled in opposite directions.

FIG. 6 is a side sectional view of a second embodiment of the present invention, where a first frame is attached to a second frame by a joint, with the first frame and second frame in a closed position.

FIG. 7 is a side sectional view of the embodiment shown in FIG. 6, with the first frame and second frame in an open position.

FIG. 8 is a top plan view of the embodiment shown in FIG. 6.

FIG. 9 is a partial plan view of the embodiment shown in FIG. 4, within a container.

### DETAILED DESCRIPTION OF THE INVENTION

The present invention is an apparatus for storing, transporting and maintaining biomaterials for use, for example, in transplantation procedures. Biomaterials such as collagen matrices sold by Ed Geistlich Sohne (Switzerland), having chondrocyte cells adhered thereto, as described in International Patent Publications WO 98/08469 and WO 00/09179, are stored, transported and maintained in an apparatus according to the present invention. The present invention includes an apparatus that stores, transports and maintains biomaterials under sterile conditions.

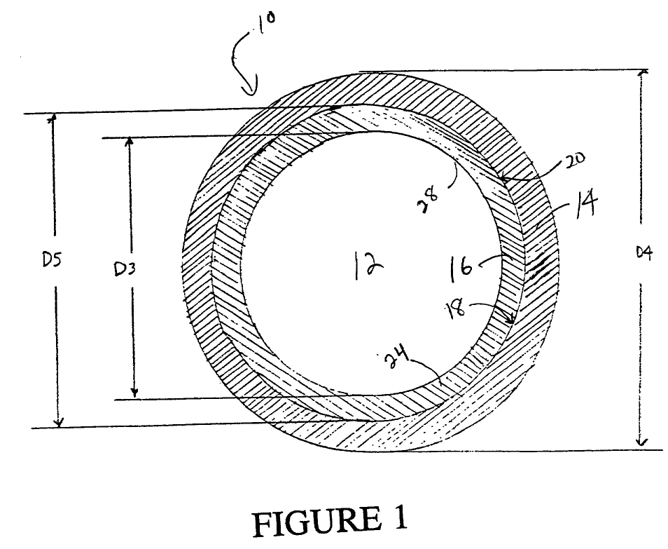

Referring to the drawings in detail, wherein like reference numerals represent like elements throughout the several figures, FIGS. 1-9 show several embodiments of the present invention for use in storing, transporting and maintaining biomaterials. An apparatus 10 according to the present invention includes a first frame 14 adjacent a second frame 16, and a center biomaterial storage area 12. The apparatus optionally includes a sterile container (See FIG. 9, and description with respect thereto) for holding first frame 14 and second frame 16.

FIG. 1 shows a top plan view of one embodiment of apparatus 10 of the present invention. A biomaterial storage area 12 is formed between a first frame 14 and a second frame 16. First frame 14 and second frame 16 are any form such that when the frames are placed adjacent to one another, a biomaterial (not shown) can be held securely between the two frames. Biomaterial storage area 12, first frame 14 and second frame 16 are of any shape, including circular, square, rectangular, triangular, hexagonal, etc., suitable for securely holding a biomaterial.

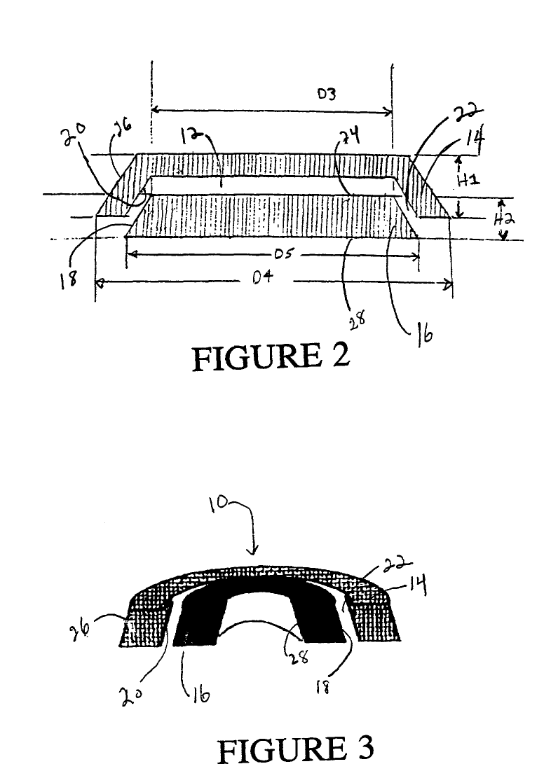

In FIG. 1, first frame 14 and second frame 16 are circular and biomaterial storage area 12 is circular. Second frame 16 is designed to fit concentrically within first frame 14 so that an outer wall 18 of second frame 16 and an inner wall 20 of first frame 14 form a space 22 in which a biomaterial is held. In use, in this embodiment, a biomaterial is placed over a top area 24 of second frame 16, and second frame 16 fits within first frame 14. The dimensions and configuration of first frame 14 and second frame 16 are designed such that a biomaterial can be securely held between first frame 14 and second frame 16 when the two frames are adjacent one another. First frame 14 and second frame 16 are lockingly engaged due to the corresponding shapes of inner wall 20 of first frame 14 and outer wall 18 of second frame 16, as shown in FIGS. 2-5. Further, an outside diameter of second frame 16 and an inside diameter of first frame 14 (both diameters shown as D5 in FIG. 1) must approximately correspond such that first frame 14 and second frame 16 interlock when holding a biomaterial. An opening represented by inside diameter (D3) of second frame 16 can have a diameter ranging from zero (that is, a closed configuration) to any desired diameter (that is, an open configuration). Where the inside diameter D3 is partially or fully closed, the closed area forms a surface upon which a biomaterial may be cut to a desired size.

As shown in FIG. 2, the outside diameter of first frame 14 is D4, and the outside diameter of second frame 16 is D5. Height H1 of first frame 14 and height H2 of second frame 16 are such that when first frame 14 and second frame 16 are placed adjacent (that is, next to, on, or partially or fully within) one another, a space 22 between first frame 14 and second frame 16 accommodates a biomaterial and holds it securely.

In one embodiment, inner wall 20 of first frame 14 forms an obtuse angle with biomaterial storage area 12, and outer wall 18 of second frame 16 is angled to correspond to the angle of inner wall 20. Outer wall 26 of first frame 14 and inner wall 28 of second frame 16 (see FIG. 1) are of any desired shape so long as outer wall 26 and inner wall 28 correspond to one another.

FIG. 3 is a partial perspective view of another embodiment of apparatus 10 according to the present invention. In this embodiment, first frame 14 fully accommodates second frame 16 and forms a space 22 for holding a biomaterial. In this embodiment, inner and outer walls of first frame 14 and second frame 16 are parallel to one another and angled to form a partially conical apparatus. In this embodiment, the cross sections of first frame 14 and second frame 16 are parallelograms. This shape allows second frame 16 to be held securely within first frame 14 when a biomaterial is within in space 22.

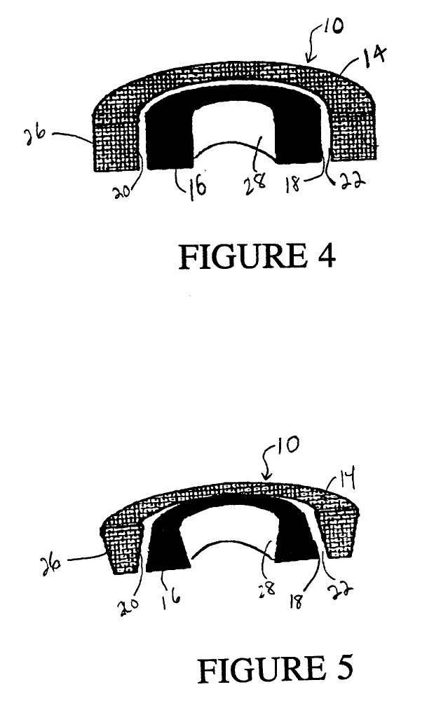

FIG. 4 is a partial perspective view of another embodiment of apparatus 10 according to the present invention. In this embodiment, the cross-sections of first frame 14 and second frame 16 are rectangular.

FIG. 5 is a partial perspective view of another embodiment of apparatus 10 according to the present invention. In this embodiment, the cross-sections of first frame 14 and second frame 16 are trapezoidal or keystone in shape. Second frame 16 is configured to fit securely within first frame 14 when holding a biomaterial in space 22. In another embodiment of the present invention (not shown), first frame 14 and second frame 16 have triangular cross-sections.

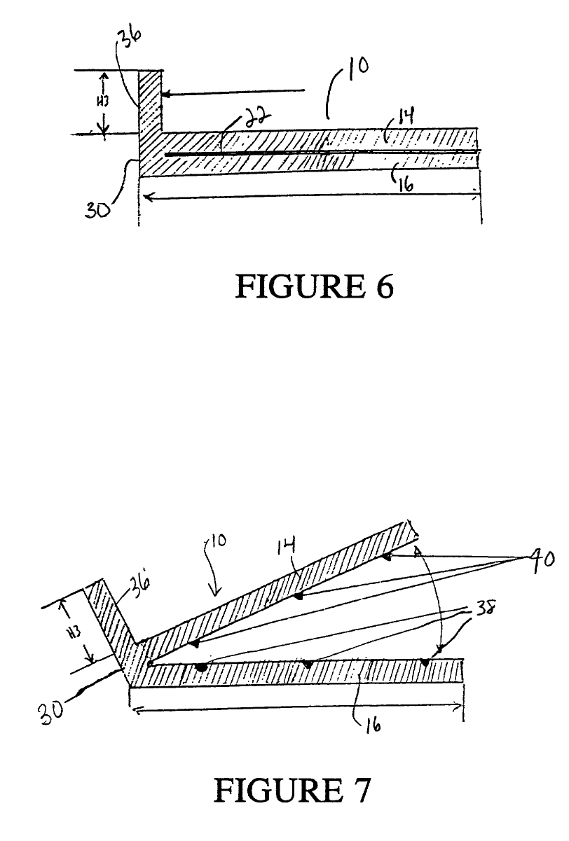

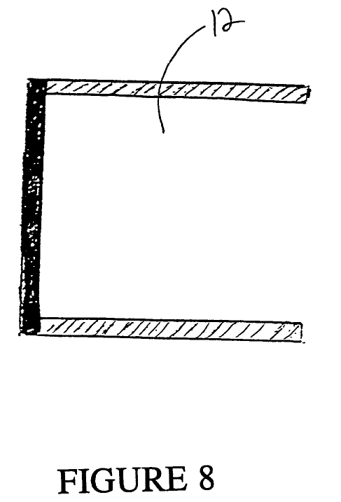

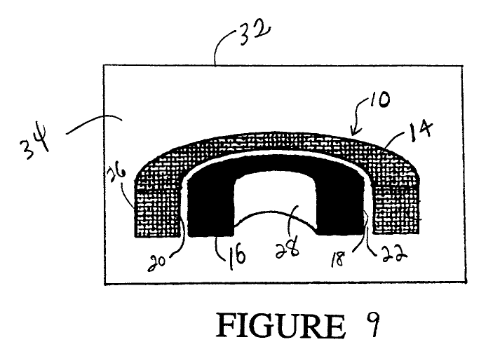

FIGS. 6-8 show another embodiment of apparatus 10 according to the present invention. In this embodiment, first frame 14 lies adjacent to second frame 16 and forms space 22 for holding a biomaterial. Biomaterial storage area 12 can be open as shown in FIG. 8 or filled depending on the particular use of apparatus 10. First frame 14 and second frame 16 are connected to one another by movable joint 30 which allows first frame 14 to move or swing relative to second frame 16, to close and open the apparatus. In one embodiment, joint 30 is a hinge or similar apparatus. In one embodiment, joint 30 is a distinct component from first frame 14 and second frame 16. Alternatively, joint 30 is integral with first frame 14 and second frame 16. In one embodiment, first frame 14 and second frame 16 are integral with one another and joint 30 is formed by folding first frame 14 over and adjacent to second frame 16. As shown in FIG. 7, in one embodiment, first frame 14 is secured to second frame 16 at a periphery of each frame by one or more indents 38 and corresponding detents 40, or any other mechanical locking mechanism, such as clips, rubber bands, or adhesives.

As shown in FIGS. 6 and 7, in one embodiment apparatus 10 includes tab structure 36 connected to first frame 14 or second frame 16 that allows a user to move first frame 14 relative to second frame 16. Tab structure 36 can have any configuration that allows a user to move first frame 14 relative to second frame 16. In this embodiment, tab structure 36 has a height H3.

FIG. 9 shows an apparatus according to the present invention, in which apparatus 10 is held within container 32 which holds, for example, a fluid media 34 for growing or maintaining a biomaterial or cells held on the biomaterial. Container 32 may be open or sealed, such as those commercially available from Nunc Gmblt (Denmark, product number D-3061) and ACO Plast A/S (Denmark, product number D-3064). Fluid media 34, for example, is cell culture or growth media.

Apparatus 10 and container 32 are made from any biocompatible material such as a biocompatible metal, rubber or plastic capable of holding but not harming a biomaterial and culture media. For example, apparatus 10 is made from stainless steel, a metal alloy, one of more polymers, ceramic, pyrolytic carbon, etc. The materials used to form apparatus 10 can be rigid or flexible.

While this invention has been described with reference to specific embodiments, it is not necessarily limited thereto. Accordingly, the appended claims should be construed to encompass not only those forms and embodiments as may be devised by those skilled in the art without departing from its true spirit and scope.

What is claimed:

1. An apparatus for storing and transporting a biomaterial comprising:
a first frame; and
a second frame adjacent to said first frame, and forming a space for holding the biomaterial between said first and second frames.

2. An apparatus according to claim 1, wherein said first frame and said second frame are lockingly engaged.

3. An apparatus according to claim 1, wherein said first frame has an inner wall and said second frame has an outer wall, and said inner wall of said first frame is adjacent said outer wall of said second frame to lockingly secure the biomaterial between said first frame and said second frame.

4. An apparatus according to claim 3, wherein said inner wall of said first frame has a first shape, and said outer wall of said second frame has a second shape, and said first shape corresponds to said second shape to lockingly secure the biomaterial between said first frame and said second frame.

5. An apparatus according to claim 1, wherein said first frame and said second frame are concentric.

6. An apparatus according to claim 1, wherein said first frame has a first cross section and said second frame has a second cross section, and said first cross section and said second cross section are trapezoids.

7. An apparatus according to claim 1, wherein said first frame has a first cross section and said second frame has a second cross section, and said first cross section and said second cross section are parallelograms.

8. An apparatus according to claim 1, wherein said first frame has a first cross section and said second frame has a second cross section, and said first cross section and said second cross section are triangles.

9. An apparatus according to claim 1, wherein said first frame lies on top of said second frame.

10. An apparatus according to claim 1, further comprising a joint joining said first frame and said second frame, allowing said first frame to move relative to said second frame.

11. An apparatus according to claim 1, wherein said first frame and said second frame are integral with one another.

12. An apparatus according to claim 1, further comprising a locking mechanism to secure said first frame to said second frame.

13. An apparatus according to claim 1, wherein said apparatus is formed from at least one biocompatible material.

14. An apparatus according to claim 1, further comprising a container surrounding said first frame and said second frame.

15. An apparatus according to claim 14, wherein said apparatus is sterile.

16. An apparatus according to claim 14, wherein said container holds fluid.

17. An apparatus according to claim 16, wherein said fluid is growth media.
